(19) United States
(12) Patent Application Publication
(10) Pub. No.: US 2009/0068687 A1
(43) Pub. Date: Mar. 12, 2009

(54) Screening platform for discovery of immunomodulatory activities in traditional medicine

(75) Inventors: Chun Lap Samuel Lo, Kowloon (HK); Man Sau Wong, Kowloon (HK); Yuen Yuen Adam Wang, Kowloon (HK)

Correspondence Address: ATTN: Maquita Wong;The Hong Kong Polytechnic University, 1800 Diagonal Road, Suite 600, Mailbox #119, Alexandria, VA, 22314, US

(73) Assignee: The Hong Kong Polytechnic University, Hong Kong (HK)

(21) Appl. No.: 11/656,421

(22) Filed: Jan. 23, 2007

Related U.S. Application Data
(63) Continuation-in-part of application No. 10/756,768, filed on Jan. 14, 2004, now abandoned.

(51) Int. Cl. G01N 33/569; C12Q 1/02; G01N 33/53; C12Q 1/26; C12Q 1/34

(52) U.S. Cl. 435/7.92; 435/29; 435/25; 435/18; 204/461

(57) ABSTRACT

The present invention relates to a method of screening traditional medicines for immunomodulating activity and anti-tumor activity. The method involves applying techniques and tools used in the field of proteomics, isolating an active ingredient from a natural ingredient historically used as a traditional medicine, stimulating splenocytes, and measuring and determining biomarker indicators resulting from such stimulation.

This application is a Continuation-in-Part of application Ser. No. 10/756,768 filed 14 Jan. 2004.

### BACKGROUND

The application of traditional medicines, such as Traditional Chinese Medicine, can be traced back to more than five thousand years. Through centuries of research and experimentation, traditional medicine practitioners have discovered thousands of plants and other natural products that have specific therapeutic effects. The therapeutic principle behind many traditional medicines is the aim to strengthen the body's immune defense system so as to attack a disease or prevent disease formation. This is based on the theory that there is always some kind of deficiency before and during the course of the disease. For example, in Traditional Chinese Medicine, Fu-zheng therapy is one of the main principles in TCM, meaning “promoting or enhancing the natural host defense mechanism” in various disease. In recent years, the positive effects of traditional medicines on the immune system have been elucidated. However, in accordance with traditional medicine practice, the effectiveness of a particular ingredient for a particular disease is unknown until results obtained from a patient are analyzed following administration of the ingredient. It is believed that a screening method applied to traditional medicines would assist practitioners in better understanding the effectiveness of particular ingredients prior to administration.

Proteomics is the study of global protein properties in a cell/tissue/organ, such as expression level, post-translational modification, protein-protein interaction, etc., on a large scale to obtain a global, integrated view of disease processes, cellular processes, and networks at the protein level. The aim of proteomics is to profile the proteins from a cellular or tissue source. This is performed by comparing diseased and healthy samples. There are currently two phases in proteomics analysis; first, all the proteins expressed by a particular organism, tissue, or cell under ‘normal’ conditions are identified and their position on two-dimensional polyacrylamide (2D) gels mapped; second, measuring the changes in the proteome that occurs in response to changing physiologic/disease conditions. In the prior art, proteomics has been applied to provide a protein profile of a cell or tissue that can be used to compare a healthy state with a disease state for protein differences in the search for drug or drug targets. Proteomics has also been applied as an assay for the potential utility of drug candidates.

Tumor necrosis factor alpha (TNF-α) was discovered in 1975 by Lloyd Old and colleagues at the Memorial Sloan-Kettering Cancer Center and was identified as an active component exhibiting antitumor activity. The major cellular source of TNF-α is the LPS-activated mononuclear phagocyte. TNF-α causes hemorrhagic necrosis of tumors in vitro, and displays cytotoxicity against cancer cells in vitro. Other biologic actions of TNF-α include its ability to cause endothelial cells to express new surface receptors that make the endothelial cell surface become adhesive for leukocytes, initially for neutrophils and subsequently for monocytes and lymphocytes, activate inflammatory leukocytes to kill microbes and activate neutrophils, and stimulate other mononuclear phagocytes to produce cytokines such as IL-1, IL-6, TNF-α, and chemokines.

Interferon (IFN) is known as a highly potent cytokine with diverse physiological functions both within and outside of the immune system. IFN-γ is secreted by thymus-derived (T) cells under conditions of activation and by natural killer cells. IFN has been known to induce the synthesis of enzymes that mediate the respiratory burst directly allowing macrophages to kill tumor cells. Other properties of IFN-γ include it being a potent activator of mononuclear phagocytes, increasing class I MHC molecule expression, enhancing both cellular and humoral immune responses in vivo, directly acting on T and B-lymphocytes to promote differentiation, promoting differentiation of CD4⁺ T cells, being one of the cytokines required for the maturation of CD8⁺ CTLs, and activating neutrophils.

Nitric oxide (NO) is a multifunctional molecule found in a variety of mammalian cells, recognized for its participation in diverse physiological and pathophysiological processes such as vascular relaxation, neurotransmission, tumoricidal and microbicidal activities, and immunosuppression. Nitric oxide is synthesized by a family of enzymes called nitric oxide synthase (NOS) using arginine as the substrate. The cytokine-inducible form, iNOS, is activated by a number of immunological stimuli, such as IFN-γ, TNF-α, and LPS, and catalyzes the high output of NO that can be cytotoxic. Previous works showed that NO released from macrophage is a mediator of microbicidal and tumoricidal activities. NO has been shown to be associated with several signal transduction mechanisms which induce apoptosis, for example, the induction of iNOS leading to NO accumulation which caused typical morphological and biochemical alterations of apoptosis, including the activation of caspase-3 and degradation of poly (ADP-ribose) polymerase (PARP).

Medicine and/or drug candidates that possess immunomodulatory activity has the ability to stimulate the production of anti-tumor cytokines such as INF-γ, TNF-α, iNOS, for example. Determination and measurement of such cytokines following application of a traditional medicine in vitro can establish potential usefulness of that medicine as an anti-tumor tool.

It is believed by the inventors herein that the application of proteomics methods and tools to traditional medicines can be used to develop powerful screening platform for traditional medicines. Such screening platform will improve the understanding of specific natural ingredients by providing information on the exact mechanism of action.

### DESCRIPTION

With a goal to acquaint the reader herewith, the inventors propose that;

This invention has as its purpose the development of a screening platform for traditional medicines,

This invention proposes achieving its purpose through the utilization of biomarkers, including but not limited to INF-γ, TNF-α, iNOS, and the methods and tools utilized in the field of proteomics,

and other purposes and methodologies, such as overcoming disadvantages and problems in the prior art.

The above statements are not intended as limitations apart from the application, but rather are to be inclusive of this application as a whole.

These and other features, aspects, and advantages of the apparatus and methods of the present invention will become better understood from the following description, appended claims, and accompanying drawings where:

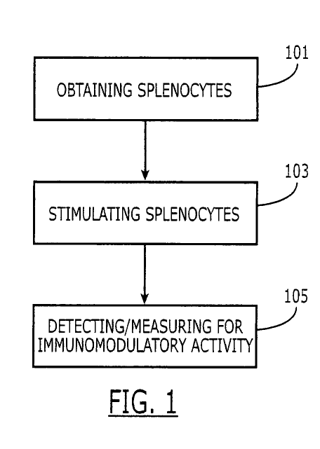

FIG. 1 shows a method determining whether a traditional medicine has anti-tumor properties in accordance with the present invention.

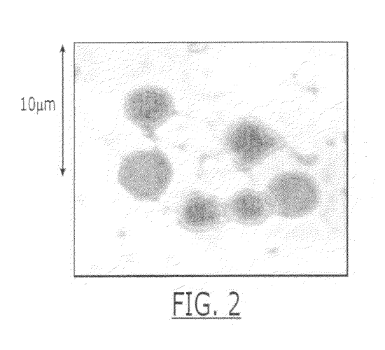

FIG. 2 shows images of isolated splenocytes obtained in accordance with the present method, pertaining to Example.

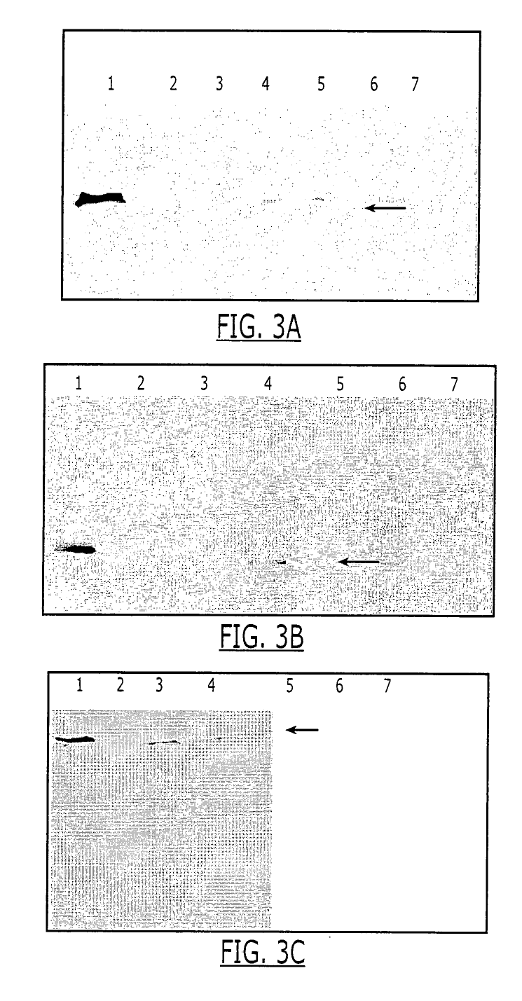

FIG. 3 shows the results of Western blotting for TNF-α, IFN-γ, and iNOS following stimulation of splenocytes with Rg1, a saponic from many herbs, for example ginseng, pertaining to Example.

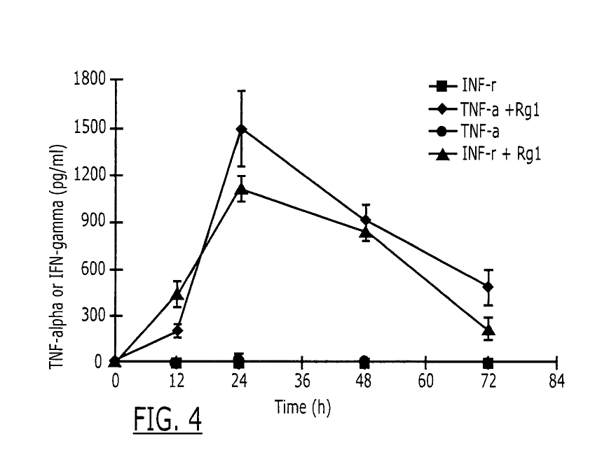

FIG. 4 shows the time course of TNF-α and IFN-γ secretion from splenocytes with and without Rg1 (5 μg/mL) treatment, pertaining to Example.

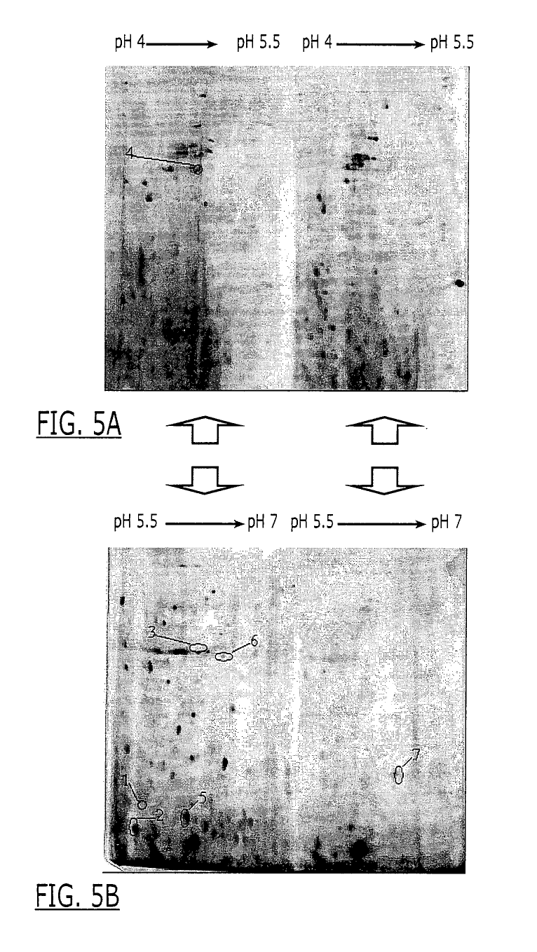

FIG. 5 shows the differential protein expression of splenocytes in the presence or absence of Rg1 compared by “Two-in-one” gel, pertaining to Example.

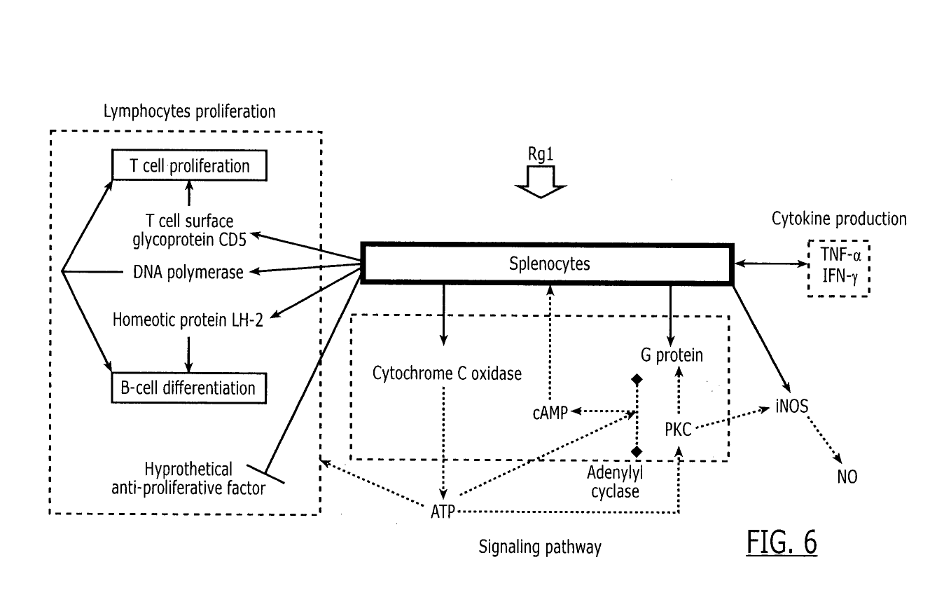

FIG. 6 shows the effect of Rg1 on protein expression in splenocytes by summarizing the effect of Rg1 on splenocytes protein expression and possible regulatory mechanism, pertaining to Example.

The following description of certain exemplary embodiment(s) is merely exemplary in nature and is in no way intended to limit the invention, its application, or uses. Throughout this description, the term “splenocyte” shall refer to all white blood cells isolated from the spleen.

The term “biomarker indicator” an indicator capable of being detected and/or measured, such indicator generally created through biological processes.

Now, specifically to FIGS. 1-6,

FIG. 1 is a method performed in accordance with the present invention, comprising the steps of obtaining splenocytes 101, stimulating the splenocytes 103, and measuring for immunomodulating activity 105. The present method combines the use of splenocytes and specific protein analyses to determine the effectiveness of a particular active agent while examining the expression of more than 100 different protein species concurrently. The present method thus allows large scale screening of natural ingredients known as traditional medicines.

### Obtaining Splenocytes 101

Splenocytes can be obtained by known protocols in the field, said protocols including the steps of obtaining a spleen, dissecting the spleen, creating a suspension with the dissected spleen, culturing cells obtained from the suspension, separating the splenocytes, and collecting the splenocytes. Spleen may be extracted from any mammal suitable for drug screening, for example rat, dog, cat, mouse, guinea pig, cattle, deer, monkey, and the like. Spleen removal may be accomplished by known splenectomy techniques, such as abdominal incision removal, laporoscopic removal, aseptic removal, and the like. Obtained spleens may then be dissected by methods such as cutting the spleen, grinding the spleen, mashing the spleen, slicing the spleen, etc. Additional tools, such as sieves, can be used for dissection. Suspensions of the spleen may then be made, for example, single cell suspensions, and then cultured. Culturing can occur by known laboratory methods including Petri dish, agar plate, microtiter plate, and batch processing methods that utilize fermentors. Culturing may occur in environments comprising growth elements such as agar, nutrients, salts, amino acids, sugars, or antibiotics; the environment may contain one or more growth elements used in conjunction with one another. In one embodiment, culturing occurs in an environment comprised of 1000 U/mL penicillin and streptomycin (PS) and 5% citric acid dextrose.

Following culturing, splenocytes may be separated out of the culture through a number of different methods, including centrifugation, gradient centrifugation, filter press, mechanical press, rotary filtration, vertical spindle separator centrifugation, horizontal bowl centrifugation, and settling and flocculation. In one embodiment, the splenocytes are isolated through the use of gradient centrifugation.

The separated splenocytes can then be collected. To ensure the splenocyte precipitate is devoid of erythrocytes, a cytolysin may be added to the precipitate. Cytolysin may be a mechanical, microbiological, or chemical addition. Lysis of erythrocytes can occur by one or more methods, including increase in temperature, extreme pH, freezing the precipitate, exposure to osmotic shock, addition of cytotoxic chemicals, such as chlorine, or addition of reagents, for example penicillin, quaternary ammonium wetting agent, or protease. In one embodiment, lysis of erythrocytes is brought about by adding ice-cold NH₄Cl solution to the precipitate.

The splenocyte can then be washed. Washing may occur through the use of media, such as minimal media, for example 1000 U/mL PS supplemented with 2 g/L sodium carbonate. In one embodiment, the resulting splenocyte is washed more than once, for example twice or three times using a minimal media.

### Stimulating Splenocytes 103

Following obtaining the splenocytes 101, the splenocytes are then stimulated using a potentially active ingredient solution. The solution is comprised of an active ingredient obtained from a natural ingredient historically used as a traditional medicine for disease prevention or treatment methods, including an herb, animal part, or mineral. “Historically” refers to the medical use of natural ingredient beginning between approximately 150 to 5000 years ago. “Traditional” refers to the reliance upon knowledge developed over centuries before the era of modern knowledge. Traditional medicine includes the fields of Traditional Chinese Medicine (TCM), ayurveda medicine, herbal medicines, homotherapies, pytotherapy, and the like. Traditional medicine is usually steeped in the culture of the society where the methods developed, for example Traditional Chinese Medicine relies upon Chinese culture in administering treatment; Ayurveda medicine is steeped in Indian culture, having been developed in India. In the traditional medicine fields, the aim is to strengthen the body's immune defense system so as to attack an illness or to prevent the formation of an illness. Following the enhancement of the body's immune system, it is then the immune system that actually attacks the illness.

Traditional medicine contrasts with modern medicines which require administered drugs and pharmaceuticals to attack the illness itself as opposed to boosting the immune system. Examples of modern medicine pharmaceuticals include antimetabolites, alkylating agents, anti-tumor agents, alkoloids, and hormones. Further, traditional medicine contrasts with modern medicine in their respective prescription methadology. Usually, traditional medicines are applied to patients, and then the patients are observed for the effect of the remedy on their well-being. Often, traditional medicines are given a shotgun prescription method, i.e., several natural ingredients are given at once, with the belief that one ingredient may be effective, or the various ingredients will work synergistically to be effective. The traditional medicines are chosen based on the practitioners understanding of the historical record about the natural ingredients. In contrast, modern medicines are first studied in detail to determine their effectiveness, such as by clinical studies, and then administered to patients in very controlled, specific manner, i.e., one drug to address a specific ailment. This contrast is likely due to the severity of modern medicine and its often side affect of killing healthy cells as well as normal cells, for example chemotherapy. In preparing the natural ingredients for consumption by a patient, non-chemically changing means are usually relied upon, for example boiling the natural ingredient, drying the natural ingredient, curing the natural ingredient, and the like. Natural ingredients are not chemically modified prior to administration.

Active ingredients obtained from a natural ingredient can include saponins, bioactive polysaccharides, immunostimulators, and the like. Active ingredients form traditional medicines can be obtained through methods including blending/soaking of the natural ingredient, heating/activating the natural ingredient, centrifuging, lyophilizing, precipitating, separating, fractionating, and testing the resultant active ingredient.

In recent years, through the process of analysis of active ingredients and testing with sensitive in vitro and in vivo models, the positive effects of natural products on the immune system and the growth of cancer cells have been elucidated. The immunomodulating activities of traditional Chinese medicine may be due to their immunostimulating polysaccharide components. Examples of sources for the active ingredient include Rhizoma bistortae, Spica prunellae, Herba bidentis bipinnatae, Folium isatidis, Rhizoma sunlacis glabrae, Radix sophorae flavescentis, Calculus bovis, Rhizoma captidis, Radix isatidis, Radix ginseng, Fructus schisaudrae, Fructus ligustin lucidi, Herba saussureae involucratae, Toitoise plastron, pipefish, Cordyceps, Colla corii asini, Placenta hominis, Herba epimedii, Ficus carica, Radix polygom multiflori, Radix angelicae sinensis, Radix morindae officinalis, Cortex eucommiae, seahorse, Glossy ganoderma, and Liquorice root.

Other sources for the active ingredient include fungi, such as poria cocos, Polyporus umbellatus, Ganoderma lucidium, G. japonicum, Cordeceps ophigoglossoides, Polyporus versicolar, Omphalia lepidescense, Dictyphora indusiata, Schizophyllum commune, and Sclerotium glucancium; plants, such as Abelmoschus glutinotextilis, Abelmoschus manihot, Acanthopanax senticosus, Aconitum camichaellii, Althaea officinalis, Althaea rosea, Angelilca acutiloba, Arctium lappa, Artemisia priniceps, Aucanacua carmizulis, Bryonia alba, Bryonia diocia, Codonopiais pilosula, Coffee sp., Coixlachryma jobi var ma yuen, Colchicum autumnale, Cucumis melo cantalupencic, Lithospermum euchromum, Nicotiana sp., Oryza sativa, Panax ginseng, Panax notoginseng, Pinus sp., Plantago major, Rumex acetosa, Saccharum officinarum, Solidago sp., Spiraea ulmaria, Trifolium pratense, Yucca schidegera, Zizyphus jujuba; medicinal plants such as Picrorhiza kurroa, Tylophora indica, Anonitum heterophyllum, Holarrhena antidysenterica, Tinospora cordifolia and Ocimum gratissium, Hemidesmus indicus, Psedustellaria heterophylla; and mixtures of plants, such as (presented in pinyin (transliteration)) xiao-chai-hi-tang, ge-gen-tang, wu-ling-tang, zhu-ling-tang, bu-zhong-yi-qi-tang, shi-quan-da-bu-tang, and ren-shen-yang-rong-tang.

Examples of active ingredients derived from natural ingredients include β(1,3)-glucan from Lentinus edodes, Schizophyllon from Shizophyllum commune, Polysaccharide K from Coriolus versicolor, GZ from Panax Ginseng, PH-1 from Pseudostellaria heterophylla, BCM from Benincas cerifera, Angelica immunostimulating polysaccharide from Angelica acutilaba, Fraction 3 from Astragalus membranaceaus, lentinum from Lentinus edodes, PH-1C from Pseudostellaria heterophylla, and Rg-1 from Panax ginseng.

In the present method, the active ingredient can be used in the active ingredient solution in an amount of from about 5 to about 20 μg/mL. In one embodiment, the active ingredient is used in an amount of 5 μg/mL. In another embodiment, Rg1 is used as the active ingredient.

The active ingredient solution also contains a saline solution, such as phosphate buffered saline (PBS), half normal saline, normal saline, quarter normal saline, or sugar in saline solution.

Stimulation of the splenocytes 103 occurs by adding the active ingredient solution to the obtained splenocytes 101 in a plate, such as a well-bottomed plate, titer plate, etc., such plate being preferably positioned in an incubator. The incubator may be set at a specific temperature, humidity, or CO₂ level. In one embodiment, the incubator controls CO₂, setting it at between 3% and 7%. In a preferred embodiment, the incubator is set at 5% CO₂ with a temperature of 37° C. Incubation can occur from between 12 and 72 hours.

### Measuring for Immunomodulating Activity 105

Following stimulation of the splenocytes by the active ingredient solution, determination and measurement for the biomarker indicators tumor necrosis factor-alpha cytokine (TNF-α), interferon gamma (IFN-γ), and inducible nitric oxide synthesis (iNOS), are performed.

Determination and measurement for immunomodulating activity can include the use of assays, for example the Bradford Protein assay, the modified Bradford Protein assay, ELISA assay, monitoring anti-body reactions, including rabbit anti-rat iNOS, rabbit anti-rat TNF-α, rabbit anti-rat IFN-γ, monoclonal anti-rat IFN-γ, monoclonal anti-rat TNF-α, biotinlated monoclonal anti-rat IFN-γ, biotinlated monoclonal anti-rat TNF-α, or other methods such as two-dimensional polyacrylamide gel electrophoresis (2DE), staining, such as silver staining, and Western Blotting. The methods of determination and measurement may be used singly, or two or more in combination in a combinatorial or sequential fashion.

The Bradford Protein assay (Bradford method) is based on the protocol recommended by the manufacturer Bio-Rad (Bio-Rad Laboratory, U.S.A.). The method involves the addition of an acidic dye coomassive blue to a protein solution, followed by subsequent measurement with a spectrophotometer. The Bradford method can be performed to determine the protein concentration of cell lysates. In accordance with protocol, following the addition of the Bradford reagent to a standard solution and protein solution, the standard and protein solutions' absorbance's can be measured at 595 nm through spectrophotometric methods.

The modified Bradford Protein assay involves the standard Bradford method, but includes the addition of an acidic dye to the protein solution, followed measurement at 595 nm with a spectrophotometer.

ELISA assays, used for detecting the presence of an anti-body or an anti-gen in a sample, can also be used. In the present method, ELISA assay can be used to study the production of IFN-γ, and TNF-α from splenocytes stimulated with the active ingredient solution. In one embodiment, ELISA assays such as the Opt EIA™ rat IFN-γ kit and the Opt EIA™ TFN-α kit, both from Pharmigen, U.S.A., can be used. The kits can be characterized as possessing both a primary and a secondary antibody, specifically a monoclonal anti-rat IFN-γ antibody can be used as the primary antibody and a biotinlated monoclonal anti-rat INF-γ can be used as the secondary anti-body, in the case of a IFN-γ kit. Alternatively, a monoclonal anti-rat TFN-α antibody can be used as the primary antibody and a biotinlated monoclonal anti-rat TFN-α as the secondary antibody, in the case of an IFN-γ kit. ELISA assays can include the use of conjugates such as avidin-HRP, and coloring substrates such as 2,2-Azin-bis-3-thylbenzthiazoline-6-sulfonic acid. Measurements using ELISA assays are made using spectrophotometric methods, usually at a wavelength of 405 using a microplate reader.

Two-dimensional Acrylamide gel Electrophoresis electrophoretogram (2-DE) analysis can be used to analyze lysate from the splenocyte. Two-dimensional polyacrylamide maps (2D) allow a means for two dimensionally comparing proteins expressed by a particular organism, tissue, or cell under “normal conditions” and proteins expressed under changing physiologic conditions. The developed 2D maps serve as a foundation on which the changes in protein expression will be analyzed.

2D electrophoresis has been the technique of choice for analyzing the protein composition of cells, tissues, and fluid, as well as for studying changes in global patters of gene expression elicited by a wide array of effectors. In 2D electrophoresis, proteins are separated in terms of their isoelectric point (pI) and molecular weight (Mw). Once 2D electrophoresis separates proteins, they can be visualized by different staining methods, for example silver staining, and protein spot profiles can be analyzed using specialized image analysis software. The proteins of interest are then selected for further characterization/identification by different identification methods.

In the present method, splenocyte lysate can be obtained by common techniques known in the art, including the steps of incubation, rinsing the splenocytes, centrifugation, lysating with a buffer, discarding of supernatant, collecting washed cells, and combining cell lysate from different animals.

Proteins can be separated based on their pI by utilizing immobilized pH gradient (IPG) strips (pH 3.9-5.1, 4.7-5.9, 3-6, 5.5-6.7, 6.3-8.3, 5-8, 7-10). Firstly, splenocytes lysates can be applied to IPG strips. Rehydration IPG of strips concurrently with sample addition before for isoelectric focusing (IEF) can be performed, IEF referring to the technique for separating different molecules by their electric charge differences. IEF can be performed at a temperature of between 20° C. and 27° C. for a period of between 14 hr to 18 hr at 50-60 v. An isoelectric instrument is suitable for use, including but not limited to Multiphor™, IPGphore™ (General Electric, U.S.A.), and a Protean IEF™ (Bio-Rad Laboratories, U.S.A.). IEF can be performed at a voltage selected from the group consisting of 500V, 1000V, 4000V, and 8000V, and at a period of between 1-2 hr.

Firstly, the strips for IEF can be equilibrated. A buffer suitable for equilibrating the strips includes 50 nM Tris-HCL equilibration buffer, pH 6.8, containing 1% (w/v) DTT, 9M urea, 30% v/v glycerol, 2% w/v SDS, and a trace amount of bromophenol blue. IEF-focused proteins can be resolved and separated by their molecular weights using sodium dodecyl sulphate polyacrylamide gel electrophoresis (SDS-PAGE). Following IEF, electrophoresis can be performed using an instrument such as a Protean III™ vertical gel electrophoresis system, or a Protean IV™ electrophoresis tank. Electrophoresis may be subjected to a current of 30 to 80 mA for about 6-16 hours.

Silver staining can be used for protein detection on the 2D gels. Through silver staining, proteins in the lysate can be detected. Silver staining methods can be modified to be compatible with matrix-assisted laser desorption/ionization-time of flight mass spectrometry (MALDI-TOF MS). MALDI-TOF MS is a technique in which a co-precipitate of a UV-light absorbing matrix and a biomolecule is irradiated by a nanosecond laser pulse. Most of the laser energy is absorbed by the matrix, which prevents unwanted fragmentation by the biomolecule. The ionized biomolecules are accelerated in an electric field and enter the flight tube. During flight, different molecules are separated according to their mass to charge ratio and reach the detector at different times. In this way, each molecule yields a distinct signal. In preparation for individual proteins for identification with the MADLI-TOF MS, gel plug containing each protein of interest (e.g. its expression was found to be up-regulated) can be picked up from the 2D gel with a plunger/cutter or any other standard technique in the art. Protein in this gel plug will be digested with enzymes, such as trypsin, chymotrypsin and/or other enzymes used in the art, by incubating with the enzyme for 12-16 hours. Peptides from the digested proteinscan be extracted by repeated and alternative addition and withdrawal of acetonitrile and 0.1% trifluoroacetic acid (TFA). The peptide extract could be speedvac to a more concentrated form and/or clean-up using tips containing ion-exchange resins (e.g. Zip-Tips). The concentrated and clean-up peptide mixture can then be mixed with the matrix. The gel matrix consists of a combination of methanol, acetic acid, formaldehyde, ethanol, acetonitrile, trifluoroacetic acid, highly purified water and acetic acid. Subsequently, through the MALDI-TOF MS, a profile of peptides (in terms of M+/Z ratios) can be obtained. This profile is called the peptide mass fingerprints (PMFs) of that protein. By comparing with PMFs generated by virtual digestion of all documented proteins in various protein and DNA databases, the protein of interest can be identified.

Besides identification methods that are MALDI-TOF MS based, methods such as Western blotting can be used. Western blotting can occur through known methods in the art, including preparation, gel electrophoresis, transferring, blocking and detecting. Detection can include calorimetric detection, chemiluminescence detection, or radioactive detection.

The above described platform provides proteomic-screening utilizing splenocytes as effectors to screen for the presence of immunomodulating activity in traditional medicine ingredient. After treating splenocytes with traditional medicine ingredient extracts, differently expressed protein species can be examined using determination and measurement methods, such as assaying, electrophoretic techniques, and mass spectrometry. Multi-targeting effects and the underlying immunomodulatory mechanism of the traditional medicine ingredient can be evaluated. This present method provides an efficient way for large scale screening of immunomodulating activities in traditional medicine ingredient.

As is known in the art, the determination and measurement of immunomodulating activity can be adjusted to meet the needs of scale-up that would be allow for large scale and fast screening of traditional medicine ingredients. Scaling up would be known to one with ordinary skill in the art to the efficiency required to effectively apply the invention

In another embodiment of the above method, biomarker indicators can also include biomarkers found in macrophages, polymorphonuclear cells, cytotoxic T lymphocytes, natural killer cells, lymphokine activated killer cells, tumor-infiltrating lymphocytes, T-lymphocytes, and B-lymphocytes.

FIG. 2-6 relate to the following example:

### EXAMPLE

Highly purified Rg1, one of the main saponins in ginseng has been shown to inhibit the growth of cancer cells. It stimulates biochemical synthesis of DNA, protein and lipid in animal tissue. Rg1 was also shown to act as an immunomodulating agent which leads to an increase in nitric oxide production in endothelial cells after Rg1 induction, an increase in T-helper activities and production of cytokine IL-1 and natural killer cell. Other properties include selectively enhanced proliferation of lymphocytes and production of IL-2. It was hypothesized that the ginsenosides' anticarcinogenic effect may correlate to the apoptotic effects initiated either by caspase 3 and/or reactive oxygen species. Despite the long list of research that studies the effects of ginseng, its molecular mechanisms of action remain largely unknown.

### Materials and Methods

### Animals

Young male Sprague Dawley rats between 8 and 10 weeks of age weighing 200 grams were provided by the animal holding center of the Department of Applied Biology and Chemical Technology, The Hong Kong Polytechnic University. All studies were conducted according to the principles and procedures detailed in the most recent publication of the NIH Guide for the Care and Use of Laboratory Animals. Animal ethics approval has been obtained from the Animal Research Subject Ethics Sub-committee of the Hong Kong Polytechnic University.

### Preparation of Rat Splenocytes

Rats were placed under deep ether anesthesia and exsanguinated via the abdominal aorta. Spleen was removed by aseptic techniques before washed twice in 10 mL of RPMI-1640 (Gibco, U.S.A.) medium containing 1000 U/mL penicillin-streptomycin (PS) (Gibco, U.S.A.). Spleens were then cut into small pieces before being sucked up and down with a plunger in a plastic syringe. Single cell suspension was prepared and placed into the same medium containing 1000 U/mL PS, and 5% acid citric dextrose. Splenocytes were then isolated by gradient centrifugation by layering the single cell suspension onto Ficoll-Paque Plus gradient (Amersham Bioscience, H.K. Ltd.) and spun at 400 g for 35 minutes at room temperature. The splenocytes-containing fraction was collected. Erythrocytes were lyzed by adding four volumes of ice-cold ammonium chloride solution (pH 7.4) at room temperature for 5 minutes and splenocytes were washed twice in plain medium. Lymphocytes were stained with Wright-Giema stain before examination under light microscope. Cell viability and number were examined after trypan blue staining under the light microscope. Splenocytes (1×10⁶) were cultured in complete RPMI-1640 medium containing 1000 U/mL PS, supplemented with 2 g/L sodium carbonate. Cells were re-suspended in the same medium with 10% FCS (Gibco, U.S.A.) and subjected to various treatments after 24 hours incubation.

Stimulation of Splenocytes with Rg1

Rg1 (dosage of 5 μg/mL) was added to splenocytes in PBS buffer in 6-well flat-bottomed plates (Nunc Maxisorp, U.S.A.) in a 5% CO₂ incubator at 37° C. Samples of conditioned media from the incubation mixture were harvested at varying time points (from 12-72 hours). These samples were kept at −80° C. until use. No sample was kept for more than 2 months. Besides Western Blotting, global protein expression in samples of Rg1 treated splenocytes (at 12 hours and 24 hours intervals) were also performed using 2-DE analysis. Cells incubated with PBS for 24 hours were used as control.

### Western Blotting and Immuno-Detection

Supernatants of splenocytes cell culture with and without Rg1 incubation were harvested at different time points and used for the detection of TNF-α and IFN-γ. Similarly, splenocyte lysate was used for the detection of expression of iNOS. The protein content of the supernatant/lysate was determined by the Bradford method (Bio-Rad Laboratory, U.S.A.). For each analysis, 350 μg of protein was analyzed by a 12.5% reducing SDS-PAGE gel ran at a constant voltage (150V) for 1 hour. Proteins were transferred onto a nitrocellulose membrane (Millipore, U.S.A.). After washing and blocking, these membranes were probed with either rabbit anti-rat iNOS (Transduction Laboratories, U.S.A.), or rabbit anti-rat TNF-α (PropTech, U.S.A.) or rabbit anti-rat IFN-γ (PropTech, U.S.A.) at a dilution of 1:500. After incubating at room temperature for 2 hours, these membranes were washed before incubating with 1:20,000 diluted goat anti-rabbit horseradish peroxidase (HRP) conjugated IgG (Sigma, U.S.A.). Positive binding was detected using the SuperSignal West Pico Chemiluminescent Kit (Pierce, U.S.A.) and the exposed X-ray films (Kodak) were scanned with a Molecular Dynamics Densitometer (Bio-Rad Laboratories, U.S.A.).

### ELISA Assay

IFN-γ as well as TNF-α ELISA assays were performed using the OptiEIATM rat IFN-γ and TNF-α kits (Pharmigen, U.S.A.). Lysates of Splenocytes with or without Rg1 stimulation were tested. In these kits, monoclonal anti-rat IFN-γ or TNF-αantibody was used as the primary antibody and biotinlated monoclonal anti-rat INF-γ or TNF-α antibody was used as the secondary antibody. After the addition of avidin-HRP conjugate, color was developed by the addition of 2,2-Azin-bis-3-thylbenzthiazoline-6-sulfonic acid and the color change was monitored at an absorbance of 405 nm with a Microplate Reader (Bio-Rad Laboratories, U.S.A.) The amount of INF-γ and TNF-α were calculated by comparing with the standard.

### 2-DE Analysis of Splenocyte Lysates

Sample Preparation

Splenocytes on culture dishes were rinsed once with RPMI-1640 after incubation with or without Rg1. Subsequently, 0.5 f/L trypsin containing 0.2 g/L EDTA was added and allowed to incubate for 3 min. Cells were then collected by centrifugation at 1,000 g for 5 minutes and washed with PBS (pH 7.2) twice. Splenocytes were then lysed with a minimal amount of lysis buffer (usually 200 μl) containing 4% Triton X-100, 9 M urea, and 1 mM PMSF for 10 minutes on ice. The supernatant was collected by centrifugation at 3,000 g for 10 minutes and protein contents were measured by a modified Bradford method using a protein assay kit by Bio-Rad Laboratories, U.S.A. Cell lysates from different animals with the same treatment were pooled together at a protein ratio of 1:1 and was used for 2-DE analysis. 120 μg of splenocytes lysate [in a buffer containing 9M Urea, 2% w/v Triton X-100, 0.5% w/v DDT (Sigma, U.S.A.), 0.4% v/v IPG buffer 4-7 (Amersham Pharmacia Biotech, Uppsala, Sweden) and trace amount of bromophenol blue to a final volume of 300 μl] was used for in-gel sample rehydration.

### 2-DE

Samples were applied onto IPG strips pH 4-7 (3×170 mm, Bio-Rad Laboratories, U.S.A.). Rehydration and subsequent isoelectric focusing was performed using either a Protean IEF Cell (Bio-Rad Laboratories, U.S.A.) or an IPGphor (Amersham Biosciences, U.S.A.). Rehydration was performed at room temperature for 16 hour at 50 V in the IPG strip holder covered with low viscosity paraffin oil. Isoelectric focusing was then performed using 500 V (1 hour), 1000 V (1 hour), 4000 V (2 hour) and 8000 V (2 hour) sequentially. Strips were then equilibrated in 50 nM Tris-HCl equilibration buffer, pH 6.8, containing 1% (w/v) DTT (Sigma, U.S.A.), 9M urea, 30% v/v glycerol, 2% w/v SDS and a trace amount of bromophenol blue for 10 minutes. After equilibration, IPG strips were transferred onto a 12.5% SDS-PAGE gels (1600×1600×1 mm) for the second dimension separation ran at a constant current of 30 mA per gel in room temperature in a Protean VI electrophoresis tank (Bio-Rad, U.S.A.). Subsequently, the gels were silver-stained with a method compatible with MALDI-TOF MS analysis. Silver-stained gels were then scanned with a molecular Dynamics Densitometer (Bio-Rad Laboratories, U.S.A.). The resulting 2-DE patterns were analyzed using the software Melanie III (Bio-Rad Laboratories, U.S.A.). An estimation of the relative quantitative changes was made on the basis of the change in percentage of volume among silver-stained protein spots. Spot changes of interest were tested on multiple gels for reproducibility.

### Protein Visualization

Silver staining was performed using a mass spectrometry compatible protocol as recommend by Bruker Daltonics (U.S.A.), manufacturer of a MALDI-TOF mass spectrometer. Briefly, a gel was fixed initially in a fixative solution containing 50% v/v methanol, 12% v/v acetic acid, 0.0375% v/v formaldehyde for 2 hours. The gel was subsequently washed with 50% methanol for another 20 minutes. The washing step was repeated twice. The gel was then oxidized in a solution containing 0.05% di-sodium thiosulphate for 1 minute followed by washing with milli-Q water (3 times, each for 20 minutes). The gel was then incubated in 0.2% silver nitrate containing 0.0375% formaldehyde for 20 minutes. The gel was again washed with milli-Q water for 3 times, each for 20 minutes. Subsequently, the gel was developed in 15% sodium carbonate solution containing 0.0375% formaldehyde and 0.004% di-sodium thiosulphate until the gel reached a desirable amount of staining. The process should not be longer than 10 minutes. Finally, the reaction was stopped with 25% methanol containing 12% acetic acid.

### Protein Identification

Differently expressed proteins from different treatment were located on the 2-DE gels either by image analysis with the software Melanie III (Bio-Rad, U.S.A.) or directly compared using the “Two-in-one” gel method as described previously (see, Wang et al. ““Two-in-one” gel for spot matching after two-dimensional electrophoresis.” Proteomics 2003, 3, 580-583). Several differently expressed protein spots were chosen and identified by peptide mass fingerprinting using a MALDI-TOF-MS (Autoflex, Bruker Daltonics, Germany). Gel plugs containing the differentially expressed protein spots were cut out from the 2-DE gels and trypsin digestion was performed (using a protocol recommended by Bruker Daltonics, Germany, which was a modification of a method described previously by Shevchenko et al. (Shevchenko et al. “Mass spectrometric sequencing of proteins silver-stained polyacrylamide gels”, Anal. Chem. 1996, 68: 850-8)). Briefly, gel plugs were cut into small pieces and washed with milli-Q water twice. They were then washed with 25 nM ammonium bicarbonate in 50% acetonitrile. Cysteine residues in the samples were reduced with 10 mM DTT and derivatized by treatment with 55 mM iodoacetamide. After acetonitrile dehydration and drying, samples were digested overnight with trypsin (5 nm/μl trypsin in 25 mM ammonium bicarbonate) at 37° C. The digested sample was extracted by sonication with 50% acetonitrile/1% trifluoracetic acid. Supernatant containing the digested peptides was collected. Digested peptides (0.5 μl to 1.5 μl) was mixed with 1-2 ml of matrix alpha-cyano-4-hydroxycinnamic acid (10 mg/ml in 50% acetonitrile, 0.1% trifluoroacetic acid) and 100 fM adrenocorticotropic hormone (ATCH) fragment 18-39 as the internal standard. The instrument was operated in the positive ion reflectron mode at 20 kV accelerating voltage. Resulting peptide mass fingerprints were searched against Swiss-Prot using the MASCOT search engine from Matrix Science. All searches were performed using a mass window between 1 and 100 kDa with a mass tolerance of +/−100 ppm. One miss cleavage per sample was allowed and cysteines were assumed to be carbamidomethylated and methionine in oxidation form.

### Results

Splenocytes Isolation

FIG. 2 shows images of splenocyte (large and small lymphocytes) isolated taken with a Leica Q500MC Image Processing and Analysis System (Leica, Germany).

Splenocytes were prepared as described in the Methods section and cells were examined after Wright-Giemsa staining. No red cell contamination was seen. Trypan blue staining confirmed that over 95% of cells isolated were viable. A small population of macrophage can also be seen attached to the bottom of the Petri dish used for culturing of the cells. As macrophages, both large and small lymphocytes were present, this preparation can be used as the effectors for studying the immunomodulatory activities of Rg1.

Effect of Splenocytes in Terms of TNF-α, IFN-γ, and iNOS Production

FIG. 3 shows results of TNF-α, INF-g, and i-NOS Western blotting upon the induction of Rg1 at different time intervals. A is for TNF-α, B is for INF-γ, and C is for iNOS.

After the successful preparation of viable splenocytes, Rg1 was added as a stimulant to study possible responses of these splenocytes. Western blotting was performed to detect the secretion of TNF-α and IFN-γ in the culture medium. Expression of iNOS was also tested in the splenocyte lysate. Most pharmacological studies of Rg1 used a concentration of 5-20 μg/ml. Preliminary studies using 5 μg/ml of Rg1 leads to a positive outcome in the TNF-α and IFN-γ experiments. Therefore, 5 μg/ml of Rg1 was used in our experiments. Splenocytes without Rg1 treatment were used as control for comparison. Results showed that neither TNF-α nor IFN-γ nor iNOS was detected in either the cell culture medium or the splenocyte lysate during 72 hours of incubation in the control. On the other hand, when stimulated with 5 μg/ml Rg1, the two cytokines and iNOS can be detected. TNF-α was detected both at 24 hour and 48 hour intervals while IFN-γ was detected at 24 hour. Inducible iNOS was detected at 12 hour and 24 hour after incubation with Rg1. Subsequently, quantitative documentation of these anticancer cytokines (TNF-α and IFN-γ) was performed using ELISA on serial samples of the culture medium.

FIG. 4 shows the assaying in triplicate of each condition.

The secretion patters of both cytokines are very similar after induction. Quantities of these cytokines increased more than 1000 fold after Rg1 induction and their levels reached a maximum at 24 hours of incubation. After 24 hours, both cytokine secretion levels started to decrease with increase in incubation time. This result is also consistent with the results from Western blot where the highest TNF-α and IFN-γ secretion is found at 24 hours after Rg1 induction.

Effect of Rg1 on Protein Expression

FIG. 5 shows the differences in protein expression between the normal sample and the Rg1 treated sample.

Since Rg1 was found to induce TNF-β and IFN-γ production after 24 hours of incubation, the effect of Rg1 on the splenocyte proteome after 24 hours of incubation was investigated. Samples with the same treatment were pooled at a protein ratio of 1:1 and used for 2-DE analysis as described in Materials and methods. All samples were collected after 24 hours of incubation either in PBS or PBS with Rg1. Sample incubated with PBS was used as the control for comparison. To facilitate identification of differently expressed protein, a “Two-in-one” gel method was performed as previously described (Wang et al., 2003). Briefly, the first dimension IEF of samples (with or without Rg1 incubation) was performed separately in different Protean IEF cell but concurrently at the same time. After the IEF was finished, the IEF stripes were cut into equal halves. Halves of the IEF strips that were corresponding to the same pH ranges but ran with different samples (with or without Rg1 incubation) were put side-by-side on top of the second dimension SDS-PAGE prepared in a Protean VI electrophoresis apparatus. The resulting gels of control and Rg1 treated samples were compared after silver staining using Melanin III. About 102 protein spots were detected from one half of the gel ran with the normal sample and 122 protein spots were detected on the other half of the gel ran with the Rg1 treated sample.

Protein Identification

MALDI-TOF mass spectrometry was used to identify differentially expressed proteins after in-gel-trypsin digestion. Seven protein spots that were differentially expressed were successfully identified by MASCOT search engine. Six proteins were up-regulated and they included cytochrome C oxidase, homeotic protein LH-2, T-cell surface glycoprotein CD5, DNA polymerase β, α-mannosidase II and guanine nucleotide binding protein G. The other protein was found to be down-regulated and it was identified as hypothetical anti-proliferative factor. Details of the 7 identified proteins are summarized in Table 1.

TABLE I
Identity of differentially expressed protein spots from splenocytes.

| | | | | % | |
|---|---|---|---|---|---|
| Spot | | Accession | Theoretical | Sequence | |
| No. | Protein name | No. | Mᵣ (kDa)/pI | coverage | Regulation |
| 1 | Homeotic | A47179 | 47.38/8.94 | 14% | Up- |
| | protein | | | | regulated |
| | LH-2 | | | | |
| 2 | Cytochrome | NP_058898 | 19.50/9.45 | 26% | Up- |
| | C oxidase | | | | regulated |
| | polypeptide | | | | |
| | IV precursor | | | | |
| 3 | DNA | AAA41900 | 38.17/8.69 | 20% | Up- |
| | polymerase | | | | regulated |
| | beta | | | | |
| 4 | Guanine | P82471 | 41.45/5.58 | 15% | Up- |
| | nucleotide- | | | | regulated |
| | binding | | | | |
| | protein G | | | | |
| 5 | T-cell | P51882; | 53.40/8.86 | 14% | Up- |
| | surface | Q63098 | | | regulated |
| | glycoprotein | | | | |
| | CD5 | | | | |
| | precursor | | | | |
| 6 | Alpha- | P28494 | 56.70/6.20 | 11% | Up- |
| | mannosidase | | | | regulated |
| | II | | | | |
| 7 | Hypothetical | I53276 | 17.74/8.39 | 33% | Down- |
| | anti-prolifer- | | | | regulated |
| | ative factor | | | | |

### Discussion

Rat splenocytes consist of macrophages, T-cells and B-cells. This population of cells was used as effectors to test for the presence of immunomodulatory activities of Rg1. With Western blotting and ELISA, it was found that Rg1 stimulated the production of TNF-α, IFN-γ and iNOS from splenocytes. TNF-α and IFN-γ production reached their peaks at 24 hours after Rg1 induction. Our results are similar to those reported by Gao et al. (Pharmaceutical Research. 1996, 13:1196-2000), whereby the highest amount of TNF-α and IFN-γ production were detected at 24 hours and 48 hours after induction with a crude preparation of Panax notoginseng on lymphocytes isolated from mouse spleens. In our experiment, a shorter time was needed for the production of TNF-α and IFN-γ to reach their peak values. It might be due to the fact that purified Rg1 instead of a crude preparation was used in our study.

On another front, the mode of action of Rg1 on a molecular level may have been revealed, which had not been discovered in previous studies. Differential protein expression in splenocytes as induced by Rg1 was studied. Based on the direct comparison of protein expression from “Two-in-one” gels, significant changes in protein expression between the normal and Rg1 treated sample was found. 87 proteins were found to be up-regulated while another 38 protein spots were found to be down-regulated. Yet another 38 spots did not have any significant apparent changes.

Differentially expressed proteins were selected and identified using MALDI-TOF mass spectrometry after trypsin digestion. Seven proteins were successfully identified in which six were up-regulated proteins. They were cytochrome C oxidase, homeotic protein LH-2, T-cell surface glycoprotein CD5, DNA polymerase beta, alpha mannosidase II and guanine nucleotide binding protein G. The remaining protein spot (hypothetical anti-proliferative factor) was found to be down regulated. From the literature, it is known that DNA polymerase-β is related to DNA synthesis. Its up-regulation indicates that Rg1 stimulates DNA synthesis. Whether this stimulation mediates through a steroid hormone-dependent or independent pathway is currently unknown. Rg1 stimulatory effects on DNA synthesis are also observed in rat and mouse bone marrow cells. On the other hand, another 2 up-regulated proteins: homeotic protein LH-2 and the T-cell surface glycoprotein CD5 are known to be involved in B-cell and T-cell proliferation. Homeotic protein LH-2 was reported as an early marker of B-lymphocyte differentiation and also involved in the control of B cell differentiation (Xu et al. Proc. Natl. Acad. Sci. U.S.A., 1993, 90:227-231). T-cell surface glycoprotein CD5, a trans-membrane protein, is known to act as a receptor in regulating T-cell proliferation (Vermeer et al. Eur. J. Immunol. 1994, 24:585-92). Another 2 up-regulated proteins that were identified: Cytochrome C oxidase and the alpha-mannosidase II might be involved in cellular metabolism processes. Cytochrome C oxidase is one of the mitochondria membrane enzymes participated in oxidative phosphorylation of the respiratory chain (Kadenbach, Biochim. Biophys. Acta. 2003, 1604:77-94). As ATP is one of the end products of respiratory process, the synthesis of ATP probably increased when cytochrome C is increased. It is known that high ATP content inside the cell contributes to energy supply for cell growth and proliferation. It also participates in transmembrane signalling pathway for cAMP production (Frizzell, Am. J. Respir. Crit. Care. Med. 1995, 151:S54-8). Alpha-mannosidase II is a Golgi membrane protein that controls conversion of mannose to complex N-glycon. It may participate in the lysosomes synthesis as proteins destined to the lysosomes are covalently modified in the Golgi body by the addition of mannose-6-phosphate. However, its exact functions are not clear.

Guanine nucleotide binding protein G is another up-regulated protein which is a membrane-associated protein that couples many types of membrane receptors to various second messenger systems (Gilman, Ann. Rev. Biochem. 1987, 56:615-49). The β and γ subunits are hydrophobic integral membrane protein that anchor the α subunit, a peripheral protein, to a plasma membrane. It is known that one component of G protein Gsα-GTP component is capable of binding to adenylyl cyclase, which stimulates the production of cAMP (Lania Eur. J. Endocrin. 2001, 145:543-559). It was reported that Rg 1 could increase intracellular cAMP and cGMP in aged animals, resulting in the expression of interleukin 2 (IL-2) and splenocytes proliferation (Liu, Yao Xue Xue Bao 1996, 31: 95-100). Thus, the up-regulation of guanine nucleotide binding protein G may imply a corresponding increase in cAMP level which in turn induced a corresponding increase in IL-2 expression and splenocytes proliferation. Hypothetical anti-proliferative factor, although its real function is not yet established, it is believed to inhibit cellular proliferation. Hence, down-regulation of this protein after Rg1 treatment indicates an increased chance of cellular proliferation.

FIG. 5 teaches possible interactions of 7 identified proteins as well as IL-2, TNF-α, IFN-γ, and iNOS in bringing out immunomodulating effects. The above results also showed that iNOS was detected at 12 hours and 24 hours after Rg1 treatment. Inducible NOS is known to produce nitric oxide that plays important roles in diverse physiological process such as vasodilatation, inhibition on platelet aggregation, and neurotransmission. Nitric oxide is also known to act as a cytotoxic mediator and contributes to the antimicrobial and tumoricidal activity of the macrophages. Thus, the up-regulation of iNOS can be used to evaluate antimicrobial activity of cells. Production of this enzyme might be regulated by the cytokines (TNF-α and IFN-γ). Karupiah et al. reported the inhibition of viral replication by IFN-g induced NOS (Karupiah et al. Science 1993, 261:1445-48). These researchers suggested that the induction of NOS is necessary and sufficient for a substantial antiviral effect of IFN-g. It was also well documented that IFN-g could act synergistically with TNF-a to promote gene expression of iNOS. Further, the increase of cytochrome C oxidase and ATP after Rg1 treatment may also increase the production of iNOS. Protein kinase C (PKC) activity is essential for iNOS expression, where PKC requires ATP in the process of binding to membrane receptor. It is also known that PKC-mediated signalling stimulates the activity of G protein. Thus, the increase in iNOS will also lead to increase in the amount of IL-2, TNF-α, and IFN-γ.

To conclude, immunomodulation is a complex activity that involves interaction between various proteins and cellular components. The use of splenocytes to screen for Rg1 immunomodulatory activity has proved to be successful in understanding the mechanisms involved in Rg1 immunomodulation. This invention may provide a framework for large-scale screening of traditional medicines.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A method of screening traditional medicines for immunomodulating activity, comprising the steps,
obtaining splenocytes;
stimulating said splenocytes with an active ingredient solution; and
determining the presence of biomarker indicators.

2. The method of claim 1, wherein said splenocytes can be obtained from animals selected from the group consisting of rat, dog, cat, mouse, guinea pig, cattle, deer, or monkey.

3. The method of claim 1, wherein stimulating said splenocytes comprises adding said active solution to said splenocyte, wherein said splenocyte is positioned in a plate.

4. The method of claim 1, wherein said active ingredient solution is comprised of an active ingredient from a natural ingredient historically used as a traditional medicine, and a saline solution selected from the group consisting of phosphate buffered saline, half normal saline, normal saline, quarter saline, and sugar in saline.

5. The method of claim 4, wherein said active ingredient can be selected from the group consisting of saponins, bioactive polysaccharides, and immunostimulators.

6. The method of claim 4, wherein said natural ingredient may come from the fields consisting of Traditional Chinese Medicine, ayurveda medicine, herbal medicines, homotherapy, and pytotherapy.

7. The method of claim 4, wherein said natural ingredient is historically used as a Traditional Chinese Medicine.

8. The method of claim 1, further comprising the step of measuring said biomarker indicator.

9. The method of claim 1, wherein said biomarker indicator may be selected from the group comprising IFN-γ, TNF-α, and iNOS.

10. The method of claim 7, wherein determining and measuring biomarker indicators can include using one or more tests selected from the group consisting of Bradford Protein assay, modified Bradford Protein assay, ELISA assay, anti-body reactions, two-dimensional polyacrylamide gel electrophoresis, staining, Western blotting, and mass spectrometry, such as MALDI-TOF MS.

11. The method of claim 1, wherein said biomarker indicators may be selected from the group consisting of homeotic protein LH-2, Cytochrome C oxidase polypeptide IV precursor, DNA polymerase beta, Guanine nucleotide-binding protein G, T-cell surface glycoprotein CD5 precursor, Alpha-mannosidase II, and Hypothetical anti-proliferative factor

12. A method of screening traditional medicines for anti-tumor activity, comprising the steps
obtaining splenocytes;
stimulating said splenocytes with an active ingredient solution;
determining the presence of biomarker indicators; and
measuring said biomarker indicators.

13. The method of claim 12, wherein said active ingredient solution is comprised of an active ingredient from a natural ingredient historically used as a traditional medicine.

14. The method of claim 13, wherein said natural ingredient is historically used as a Traditional Chinese Medicine.

15. The method of claim 12, wherein said biomarker indicator may be selected from the group comprising IFN-γ, TNF-α, and iNOS.

16. The method of claim 12, wherein determining and measuring biomarker indicators can include using one or more tests selected from the group consisting of Bradford Protein assay, modified Bradford Protein assay, ELISA assay, anti-body reactions, two-dimensional polyacrylamide gel electrophoresis, staining, Western blotting, and mass spectrometry, such as MALDI-TOF MS.

17. A method of screening Traditional Chinese Medicines for anti-tumor activity, comprising the steps
obtaining splenocytes;
stimulating said splenocytes with an active ingredient solution containing an active ingredient from a natural ingredient historically used as a Traditional Chinese Medicine;
determining the presence of biomarker indicators; and
measuring said biomarker indicators.

18. The method of claim 17, wherein said biomarker indicator may be selected from the group consisting of IFN-γ, TNF-α, and iNOS.

19. The method of claim 17, wherein determining and measuring biomarker indicators can include using one or more tests selected from the group consisting of Bradford Protein assay, modified Bradford Protein assay, ELISA assay, anti-body reactions, two-dimensional polyacrylamide gel electrophoresis, staining, Western blotting, and mass spectrometry, such as MALDI-TOF.

20. The method of claim 17, wherein said biomarker indicators may be selected from the group consisting of homeotic protein LH-2, Cytochrome C oxidase polypeptide IV precursor, DNA polymerase beta, Guanine nucleotide-binding protein G, T-cell surface glycoprotein CD5 precursor, Alpha-mannosidase II, and Hypothetical anti-proliferative factor
